SEQUENCE LISTING (9 sequences)

SEQ ID NO: 1 - RNA, 22 nt, Homo sapiens
  microRNA
aaaccguuaccauuacugaguu

SEQ ID NO: 2 - RNA, 72 nt, Homo sapiens
  microRNA
cuugggaauggcaaggaaaccguuaccauuacugaguuuaguaaugguaaugguucucuugcuauacccaga

SEQ ID NO: 3 - RNA, 21 nt, Homo sapiens
  microRNA
aggcaagaugcuggcauagcu

SEQ ID NO: 4 - RNA, 71 nt, Homo sapiens
  microRNA
ggagaggaggcaagaugcuggcauagcuguugaacugggaaccugcuaugccaacauauugccaucuuucc

SEQ ID NO: 5 - RNA, 22 nt, Caenorhabditis elegans
  microRNA
ucaccggguguaaaucagcuug

SEQ ID NO: 6 - RNA, 21 nt, Artificial Sequence
  siRNA sense strand
agguguaucuccuagacactt

SEQ ID NO: 7 - RNA, 21 nt, Artificial Sequence
  siRNA antisense strand
gugucuaggagauacaccutt

SEQ ID NO: 8 - RNA, 21 nt, Artificial Sequence
  siRNA sense strand
gagauuacgauugcugggctt

SEQ ID NO: 9 - RNA, 21 nt, Artificial Sequence
  siRNA antisense strand
gcccagcaaucguaaucuctt

(19) United States
(12) Patent Application Publication
(10) Pub. No.: US 2021/0238552 A1
(43) Pub. Date: Aug. 5, 2021

(54) EXTRACELLULAR VESICLES LOADED WITH AN EXOGENOUS MOLECULE

(71) Applicant: UNICYTE EV AG, Oberdorf (CH)

(72) Inventors: Giovanni CAMUSSI, Torino (IT); Margherita Alba Carlotta POMATTO, Cavagnolo (Torino) (IT)

(73) Assignee: UNICYTE EV AG, Oberdorf (CH)

(21) Appl. No.: 17/267,086

(22) PCT Filed: Aug. 5, 2019
(86) PCT No.: PCT/EP2019/070957

§ 371 Date: Feb. 9, 2021

(30) Foreign Application Priority Data
Aug. 10, 2018 (EP) 18188493.3

(51) Int. Cl. C12N 5/0775; C12N 5/071; C12N 15/113; C12N 15/89

(52) U.S. Cl. CPC C12N 5/0663; C12N 5/0672; C12N 2320/32; C12N 15/895; C12N 2310/141; C12N 15/113

(57) ABSTRACT

Active loading of extracellular vesicles (EVs) with an exogenous molecule without damaging the extracellular vesicles is provided. A composition comprising a population of extracellular vesicles loaded with an exogenous molecule, which have maintained their integrity, original endogenous cargo and functionality, compared to unloaded controls is also provided. Extracellular vesicles are derived from a stem cell, preferably an adult stem cell, or from a biological fluid, a conditioned cell medium or a tissue culture medium.

Extracellular vesicles (EVs) are a heterogeneous population of particles released by virtually all living cells. They mainly include microvesicles, released through the budding of the plasma membrane, and exosomes, derived from the endosomal compartment. EVs are fundamental players in the cell-cell communication by being natural carriers of a complex cargo which includes proteins, lipids and nucleic acids. The discovery of EVs as natural vehicles of functional nucleic acids has raised great interest in their use as drug delivery carriers. In recent years, several evidences demonstrated the feasibility of engineering EVs to deliver nucleic acids, particularly RNA, to target cells as therapeutic molecules for the treatment of diseases in which specific genes are overactive, such as cancer. Various methods to load nucleic acids in isolated EVs have been investigated, including passive loading such as co-incubation and hypotonic dialysis. However, the presence of the lipid bilayer membrane restricts the passive migration of large hydrophilic compounds such as nucleic acids into EVs, and entails low encapsulation efficiency. In addition to passive loading methods, approaches for active loading of EVs with nucleic acids have been developed, including electroporation, sonication, transfection, extrusion and saponin permeabilization. Among these methods, electroporation has been largely exploited because during this process, after stimulation with an electrical signal, the lipid molecules of the plasma membrane shift positions and open up transient pores that serve as the conductive pathway for outside molecules to pass through. Despite initial promising results, significant drawbacks have been reported in connection with the use of electroporation as a RNA loading method of EVs. In fact, a significant variability has been observed across electroporation experiments carried out under same experimental conditions. Moreover, electroporation of EVs with siRNA has been shown to be accompanied by extensive formation of siRNA insoluble aggregates, which in turn leads to a drastic decrease in the efficiency of nucleic acid encapsulation into the EVs (Kooijmans S A et al., Electroporation-induced siRNA precipitation obscures the efficiency of siRNA loading into extracellular vesicles. J Control Release 2013; 172:229-38). In addition, electroporation may trigger aggregation or fusion of EVs themselves, thereby causing the generation of severely damaged EVs following nucleic acid loading (Banizs A B et al., In vitro evaluation of endothelial exosomes as carriers for small interfering ribonucleic acid delivery Int J Nanomedicine. 2014; 9: 4223-4230).

WO10119256 describes the use of electroporation and transfection as methods suitable for loading small size extracellular vesicles, in particular exosomes derived from immature dendritic cells, with genetic material. The loaded exosomes can be used for in vivo delivery of genetic material. However, WO10119256 indicates that it is difficult to come up with a suitable electroporation protocol for exosome loading with nucleic acid due to inconsistencies of transformation results.

EP2010663B1 discloses methods of delivering nucleic acid constructs by exosomes to recipient cells. Genetically modified exosomes are produced by introducing constructs of RNA or DNA directly into these small size vesicles by using conventional techniques such as in vitro transformation, transfection, and microinjection.

US2014356382A1 describes the use of exosomes as delivery vehicles for protein and/or peptide biotherapeutics, including antibodies. The exogenous protein and/or peptide may be introduced into the exosomes by electroporation or the use of a transfection reagent.

Wahlgren J et al., Plasma exosomes can deliver exogenous short interfering RNA to monocytes and lymphocytes, Nucleic Acids Res. 2012 40(17):e130, discloses that plasma exosomes can deliver exogenous siRNA to recipient monocytes and lymphocytes causing selective silencing of MAPK-1 target gene. Although a electroporation protocol has been optimized for transferring siRNA into exosomes, Wahlgren et al., acknowledges that the exosomes loading with nucleic acids by electroporation lacks reproducibility since transfection electroporation efficiency is drastically varying across experiments while the experimental settings are maintained the same.

To date, various procedures for loading EVs with exogenous molecules have been established, which however suffer from severe technical limitations, more particularly a low encapsulation efficiency along with integrity and functionality damage of generated loaded vesicles.

There is therefore the need of achieving efficient and well-controlled loading of extracellular vesicles with exogenous molecules without affecting the structure and/or the biological activity of these vesicles.

In particular, there is the need of providing extracellular vesicles which, after efficient loading with exogenous molecules, preserve their structural integrity, particularly with regard to size and membrane protein composition.

There is also a need of providing extracellular vesicles loaded with exogenous molecules which at the same time have retained their original content of endogenous molecules, without any significant loss in molecules composition and abundance.

There is further a need for a method enabling efficient loading of exogenous molecules into extracellular vesicles by causing minimum damage to these vesicles

These and other needs are met by the composition comprising a population of exogenous molecule-loaded extracellular vesicles (EVs) as defined in the appended claim 1 and by the method as defined in the appended claim 23. The dependent claims define preferred features of the composition and method of the invention and form an integral part of the description.

As further illustrated in the Experimental Section below, the present inventors have surprisingly succeeded in obtaining extracellular vesicles efficiently loaded with an exogenous molecule without causing any damage to these vesicles. More particularly, the absence of damage in the population of EVs following loading was assessed by the present inventors by evaluating three different parameters, namely EVs integrity, the capability of retaining the original content of endogenous molecules and the maintenance of EVs membrane protein composition. As controls, unloaded extracellular vesicles were used.

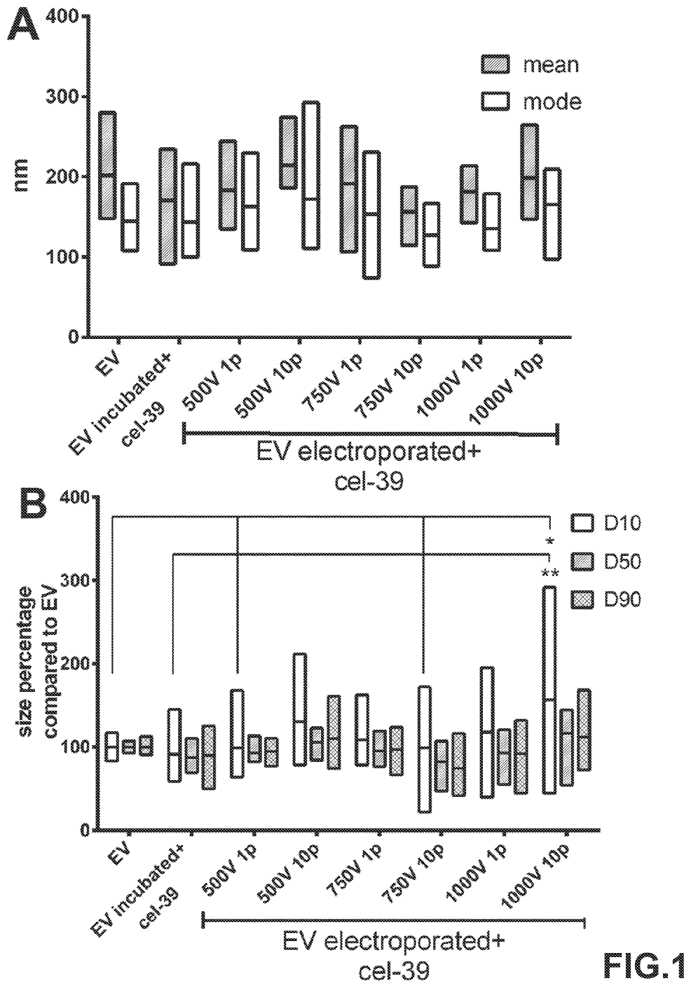

As a first step, the structural integrity of EVs loaded with an exogenous molecule was verified by quantifying EVs number and size via nanoparticle tracking analysis (NTA). The present inventors found that, even though the NTA profiles obtained with loaded EVs were similar to the profiles generated with unloaded vesicles, significant changes in the diameter distribution in the populations of loaded EVs occurred depending on the experimental conditions used in the loading process. In particular, under determined loading conditions, an increase in the maximum diameter size was observed in 90% of the EVs population loaded with an exogenous molecule, compared to unloaded EVs (FIGS. 1, 2, 10 and 11). Such an increase in diameter size may be ascribed to vesicles aggregation, which is a clear indication of vesicles structural damage. By analyzing the EVs which maintained their morphology after exogenous molecule loading, i.e. no detectable variation in the maximum diameter size in 90% of the population, the present inventors measured the increase as percentage of the mean diameter in the populations of intact loaded EVs compared to unloaded controls (Table 2). The highest increase in the mean diameter size in this EVs population, corresponding to the value of 10% measured in the MSC-EVs 2p population, was determined as a threshold above which the exogenous molecule-loaded EVs population is to be considered as damaged, i.e. showing altered EVs size and vesicles aggregation.

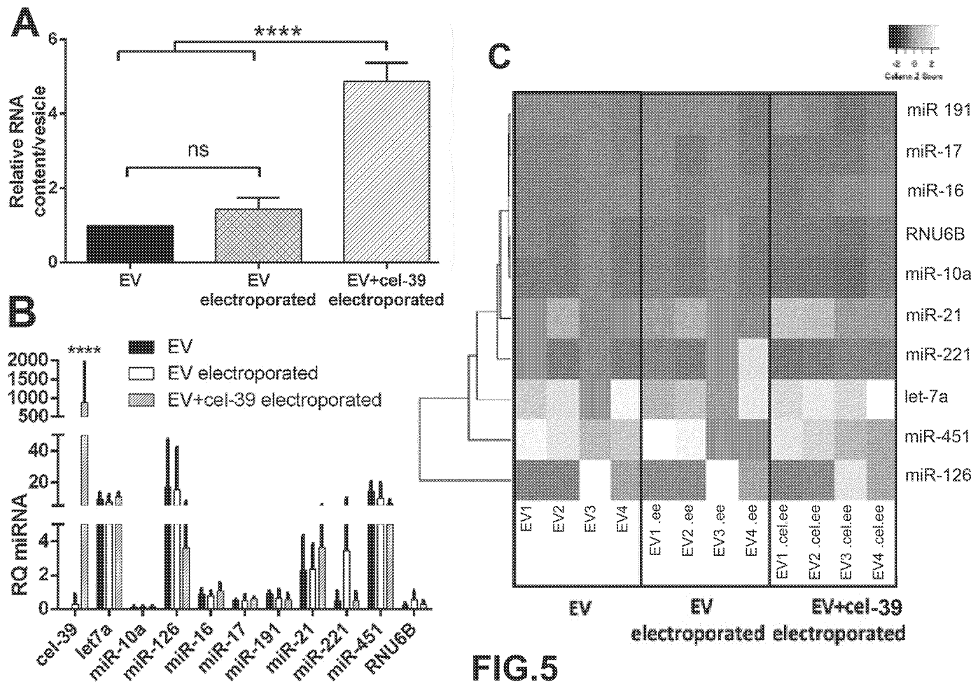

In order to further characterize the absence of damage in EVs loaded with exogenous molecules, the present inventors verified whether, besides structural integrity, the employed loading technique preserved the EVs original endogenous nucleic acid content. Indeed, the increase in membrane permeability caused by loading methods such as electroporation, which induce the formation of transient pores in the lipid bilayer, may lead to the leakage of molecules enclosed within these vesicles. As illustrated in FIG. 5, the analysis carried out by the present inventors did not detect any significant variation in the total RNA content, quantified by spectrophotometry, as well as in the expression profile of a panel of representative miRNAs, in the population of EVs after exogenous molecules loading, as compared to the unloaded EVs.

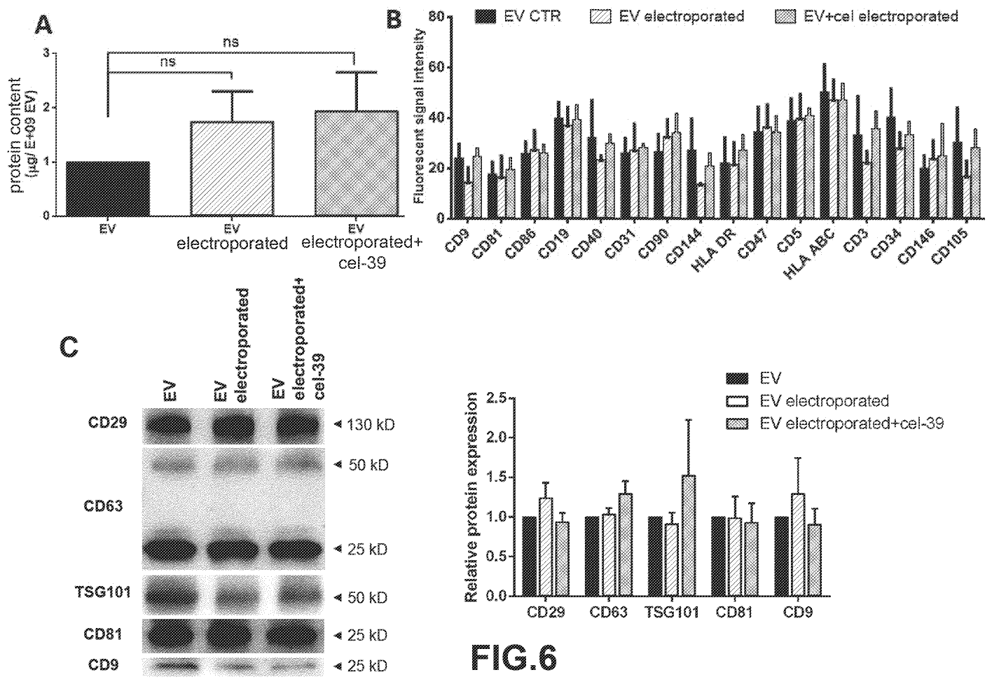

Moreover, FACS analysis carried out by the present inventors showed no significant degradation of surface protein markers in the loaded EVs, demonstrating that these vesicles retain the original membrane proteins composition and distribution (FIG. 6B). Membrane-bound proteins are known to play a fundamental role in determining EVs fate and functions, and qualitative and/or quantitative alterations in their content represent an evidence of damaged EVs. In the course of this study, the present inventors analyzed a panel of surface proteins reported as highly expressed in the EVs and measured the reduction in expression level for each marker in the population of exogenous molecule-loaded EVs as compared to the population of unloaded extracellular vesicles. By calculating the average across these measurements, the present inventors identified a threshold value corresponding to 15% above which the reduction in the expression level of surface markers is indicative of damaged EVs.

Thus, one aspect of the present invention is a composition comprising a population of extracellular vesicles (EVs), characterized in that the EVs in the population are loaded with an exogenous molecule and are not damaged, wherein the absence of damage is defined as follows:

(i) the mean diameter in the population of exogenous molecule-loaded EVs is increased by not more than 10% as compared to the mean diameter in a population of unloaded control EVs;

(ii) the total nucleic acid content present in the population of exogenous molecule-loaded EVs is not significantly decreased as compared to the total nucleic acid content present in the population of unloaded control EVs; and/or

(iii) the mean expression level of a panel of surface markers in the population of exogenous molecule-loaded EVs is reduced by not more than 15% as compared to the mean expression level of the same panel of surface markers in the population of unloaded control EVs.

Unless defined otherwise, all technical and scientific terms used herein have the same meanings as commonly understood by one of ordinary skill in the art to which this invention belongs.

The term “significantly” as used herein means that there is a statistically significant difference between loaded EVs and unloaded control EVs as evaluated by setting the statistical significance level at 0.05 (p-value<0.05). Within this context, statistical analysis was conducted by using any of the known suitable statistical methods known in the prior art such as ANOVA and t-test.

The term “EV cargo” as used herein refers to the nucleic acid, protein and lipid content naturally enclosed within the extracellular vesicle. The nucleic acid cargo of EVs comprises typically microRNAs (miRNAs), intact mRNA molecules or fragments thereof, particularly 3′ UTR mRNA fragments, ribosomal RNA (rRNA), fragments of tRNA, long non-coding RNA, piwi-interacting RNA, vault- and Y-RNA.

The term “exogenous molecule” as used in the present description encompasses both a heterologous molecule which is not part of the natural cargo of the EVs population of the invention as such and a molecule which is normally part of the endogenous cargo of the EVs population of the invention as such. It is therefore to be intended that the loading of EVs with an exogenous molecule, such as for example a nucleic acid molecule, which is normally part of their endogenous cargo leads to a population of EVs according to the invention enriched for this specific molecule.

The term “heterologous molecule” as used herein refers to a molecule, for example a nucleic acid molecule, derived from another animal or vegetal species or from another organ or tissue than the population of extracellular vesicles according to the invention, or from different donor cells, different conditions, or from genetically modified donors cells.

In accordance to the present invention, the “exogenous molecule” may be a nucleic acid, a protein, a peptide, an aptamer, a chemical drug, or any combination thereof.

Examples of nucleic acid molecules which may be introduced into the EVs according to the invention include, but are not limited to, DNA fragments, DNA plasmids, mRNA, tRNA, rRNA, miRNA, siRNA, long and short non-coding RNA, regulating RNA, and antisense RNA. The nucleic acid to be loaded into the population of EVs according to the invention is chosen on the basis of the desired effect of this genetic material, particularly on the effect on the cell into which it is intended to be delivered. For example, the nucleic acid may be useful in gene therapy, for example in order to express a desired gene in a cell or group of cells. Such nucleic acid is typically in the form of plasmid DNA or viral vector encoding the desired gene. Nucleic acids can also be used in gene silencing. The latter may be useful in clinical therapy to switch off aberrant gene expression or in animal model studies to create single or more genetic knock outs. Typically such nucleic acid is provided in the form of siRNAs.

Where the exogenous molecule loaded into the EVs according to the invention is a protein, such protein may be, for example, a growth factor, a cytokine, a receptor, a recombinant protein or an antibody.

Exemplary drugs which may be loaded into the EVs according to the invention include, but are not limited to, anticancer agents, anti-inflammatory agents, angiogenesis inhibitors, regenerative molecules.

Extracellular vesicles are produced by many different cell types—so-called donor cells—and are ubiquitously present in biological fluids, and cellular or tissue cultures. Thus, in accordance with the present invention, the EVs in the population of the invention can be obtained from any suitable cell type, preferably from a nucleated mammalian cell, more preferably from a stem cell, even more preferably from an adult stem cell.

Within the context of the present description, the expression “adult stem cell” is intended to mean a stem cell that is isolated from an adult tissue, in contrast with an “embryonic stem cell” which is isolated from the inner cell mass of a blastocyst. Adult stem cells are also known as “somatic stem cells”.

In a preferred embodiment of the present invention, the adult stem cell is a human liver stem cell or a human mesenchymal stem cell.

A preferred human liver stem cell is the human non-oval liver stem cell (HLSC) expressing both mesenchymal and embryonic stem cell markers. HLSCs are disclosed e.g. in WO2006126236.

In another preferred embodiment, the human mesenchymal stem cell is derived from human adult bone marrow (BM-MSC).

According to another embodiment of the invention, the EVs in the population of the invention are derived from a biological fluid or from a conditioned cell or tissue culture medium.

As known by those skilled in the art, conditioned medium is spent media harvested from cultured cells or tissues. It contains metabolites, growth factors, and extracellular matrix proteins secreted into the medium by the cultured cells. Within the context of the present invention, the culture medium may be conditioned by a stem cell, preferably by an adult stem cell.

In a more preferred embodiment, the EVs according to the invention are derived from a biological fluid selected from whole blood, plasma, serum or urine.

There are various methods for isolating EVs from biological fluids, cell cultures or tissues supernatants, examples of which include, but are not limited to, density gradient, ultracentrifugation, filtration, dialysis and freeflow electrophoresis.

According to the present invention, the EVs in the population are actively loaded with the exogenous molecule after vesicles isolation or purification from donor cells. Such approach, typically referred to as ex-vivo loading, differs substantially from the in-vivo approach wherein the exogenous molecules are pre-loaded in the donor cells before EVs isolation.

Preferably, the expression level of surface markers in the population of EVs according to the invention is reduced by no more than 12% as compared to the expression level of surface markers in the control population of unloaded extracellular vesicles.

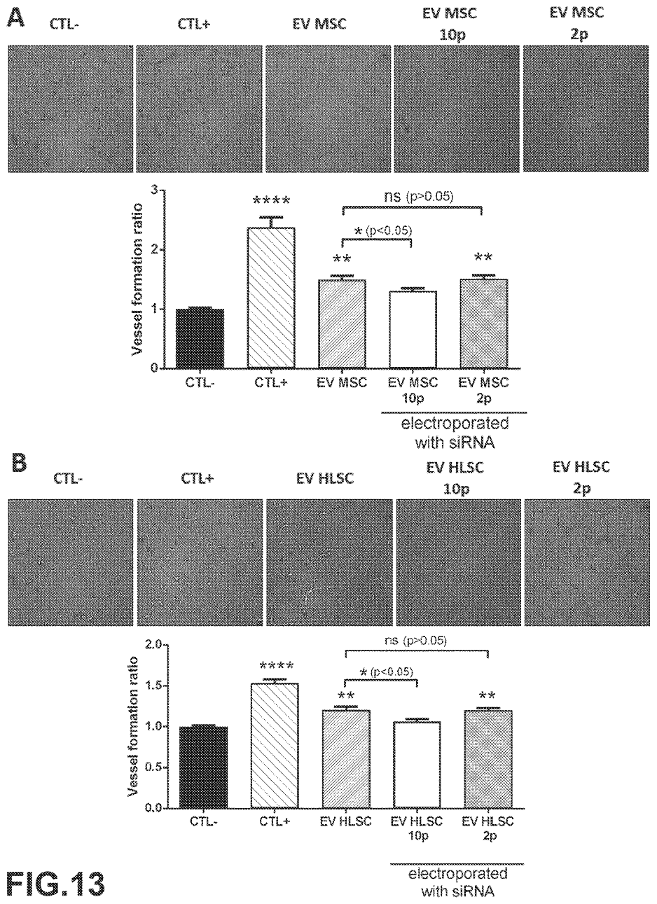

Extracellular vesicles display versatile biological functions and play important roles in different physiological and pathological processes, such as cancer and autoimmune diseases. Further supporting the absence of damage following EVs loading, the present inventors have verified that the biological properties of the population of EVs according to the invention are significantly preserved after loading with an exogenous molecule as compared to the population of unloaded EVs. Functional evaluation was carried out by assessing the loading effects on the pro-angiogenic activity which is naturally promoted by extracellular vesicles. As illustrated in FIG. 13, in an in vitro model of angiogenesis, exogenous molecule-loaded EVs are capable to induce vessels formation of endothelial cells at the same rate as unloaded EVs, thereby indicating the absence of functional damages in the population of EVs according to the invention.

Therefore, in a preferred embodiment of the invention, the biological activity in the population of EVs according to the invention is not significantly reduced as compared to the biological activity in the population of unloaded extracellular vesicles.

In a more preferred embodiment of the invention, the biological function retained by the population of EVs according to the invention is a pro-angiogenic activity. A pro-angiogenic activity is demonstrated by an increase in vessel formation by at least 10%. according to a tubulogenesis assay.

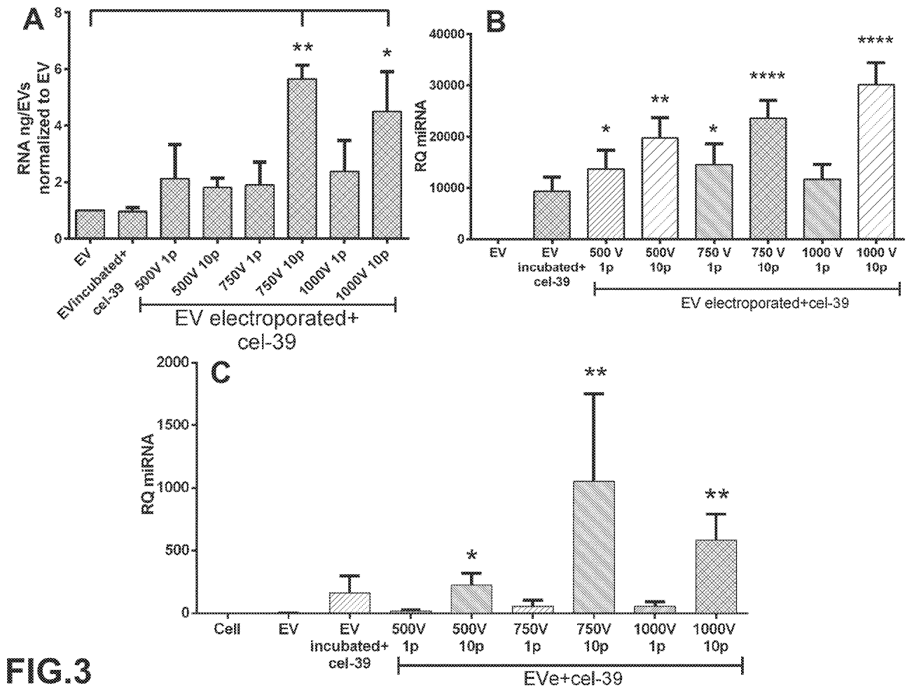

The population of EVs according to the invention has retained structural integrity and functionality following loading by electroporation, with concomitant incorporation of high levels of exogenous molecules (FIGS. 3, 4 and 9).

In one preferred embodiment, the present invention provides a population of extracellular vesicles (EVs) which are loaded with an amount of exogenous molecules of at least 3 ng/10¹⁰ EVs, more preferably with an amount of exogenous molecules of at least 2 ng/10¹⁰ EVs.

The population of EVs according to the invention may be loaded with a nucleic acid, preferably with a microRNA (miRNA) or a small interfering RNA (siRNA). In this embodiment, the loaded miRNA and/or siRNA may provide a new functional activity to the EVs or increase an already present EVs activity.

In a one embodiment of the present invention, the loaded miRNA and/or siRNA exhibits pro-angiogenic activity, for example by driving the migration and formation of new vessels in recipient cells, preferably in endothelial cells.

In another embodiment of the present invention, the loaded miRNA and/or siRNA function as anti-cancer agents by acting, for example, as pro-apoptotic and/or anti-proliferative molecules.

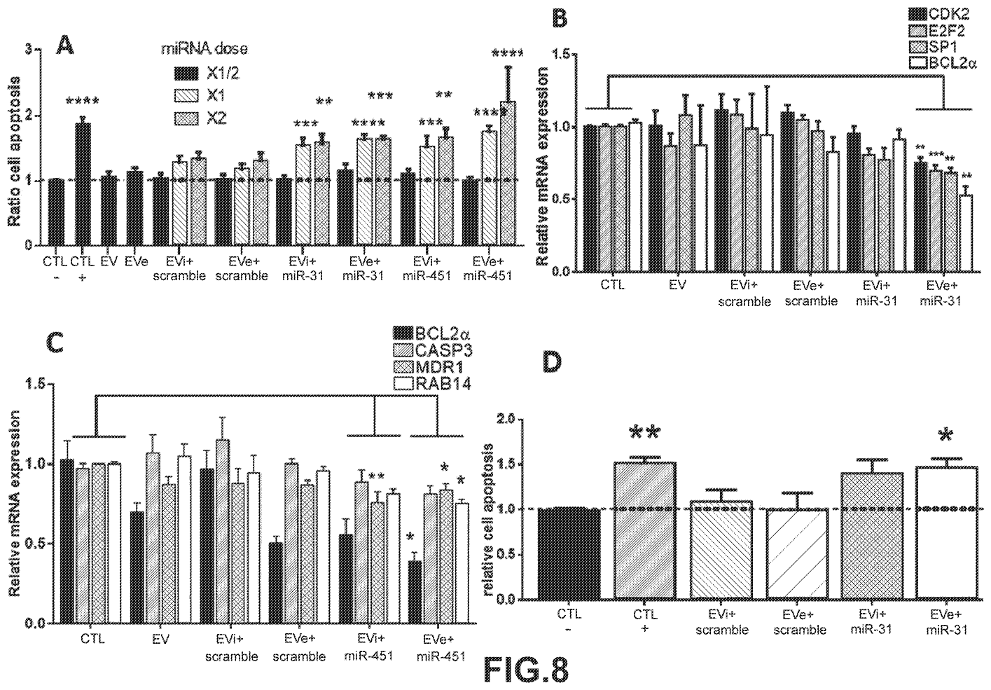

Accordingly, the population of EVs of the invention may act as carrier to effectively deliver loaded miRNA and/or siRNA into recipient cells, for example into endothelial cells, immune cells, hematopoietic cells or cancer cells. The miRNA and/or siRNA cargo may be delivered by the EVs population of the invention to recipient cells through cell attachment via surface adhesion proteins or by directly fusing to the plasma membrane. As illustrated in FIG. 8, the present inventors have shown that the EVs of the invention loaded with exogenous antitumor miRNA molecules are able to induce a significant pro-apoptotic effect on hepatoma cells.

In a particular preferred embodiment, the loaded miRNA is hsa-miR-451 or hsa-miR-31, more preferably hsa-miR-451a and/or hsa-miR-31-5p.

As used herein, the term “hsa-miR-451” encompasses
hsa-miR-451a (SEQ ID NO. 1: 5′AAACCGUUACCAUUACUGAG
UU 3′)
and
hsa-miR-451a stem-loop sequence (SEQ ID NO. 2: 5′C
UUGGGAAUGGCAAGGAAACCGUUACCAUUACUGAGUUUAGUAAUGGUAAU
GGUUCUCUUGCUAUACCCAGA 3′).
As used herein, the term “hsa-miR-31”” encompasses
hsa-miR-31-5p (SEQ ID NO. 3: 5′AGGCAAGAUGCUGGCAUAG
CU 3′)
and
hsa-miR-31 stem-loop sequence (SEQ ID NO. 4: 5′GGA
GAGGAGGCAAGAUGCUGGCAUAGCUGUUGAACUGGGAACCUGCUAUGCCA
ACAUAUUGCCAUCUUUCC 3′).

EVs loaded with siRNA molecules may be used for the therapeutic treatment of genetic diseases, by silencing aberrant genes. For instance, siRNA loaded EVs may be employed for the treatment of familial hypercholesterolemia, Huntington's Disease, autoimmune disorders, viral infections, degenerative processes and diabetes. Additionally, EVs loaded with miRNAs may restore the miRNA dysregulation typical of several conditions, including cancer, autoimmune diseases, diabetes, organ injury, fibrotic diseases as well as promote regenerative processes. Moreover, targeted miRNAs delivered to recipient cells by loaded EVs may restore altered angiogenesis, which is a common mechanism in the pathogenesis of several disorders, including cancer, stroke and coronary heart disease.

Therefore, nucleic acid-loaded EVs according to the invention may be used for the treatment of cancer diseases, cardiovascular diseases, genetic diseases, fibrotic diseases, wound healing, organ injury and viral infections. In particular, the population of EVs of the invention may be used for the treatment of stroke and coronary heart disease. The population of EVs of the invention may also be used for the treatment of hypercholesterolemia, particularly familial hypercholesterolemia, autoimmune disorders, degenerative processes and diabetes.

A panel of EVs surface markers comprises proteins which are known to be incorporated in the membrane of these vesicles, influencing EVs biodistribution. Preferably, the panel of EVs surface markers according to the invention comprises one or more markers selected from the group consisting of CD9, CD19, CD81, CD86, CD90, HLA DR, CD47, CD34, CD40, CD31, CD144, CD3, CD146, CD105, CD5, HLA ABC, CD29, CD44, CD49d, CD49e, CD49f. In one embodiment, the panel of surface markers according to the invention comprises CD9, CD19, CD81, CD86, CD90, HLA DR, CD47, CD34, CD40, CD31, CD144, CD3, CD146, CD105, CD5, HLA ABC and any combination thereof. In another embodiment, the panel of surface markers according to the invention comprises CD29, CD44, CD90, CD105, HLA-ABC and any combination thereof. In yet another embodiment, the panel of surface markers according to the invention comprises CD29, CD44, CD49d, CD49e, CD49f, HLA-ABC and any combination thereof.

The active ex-vivo loading of exogenous molecules into the population of EVs according to the present invention may be accomplished by a number of different technique known in the art, including, for example, electroporation, sonication, lipofectamine mediation, microinjection, co-incubation, dialysis and freeze-thaw cycles.

In a particularly preferred embodiment of the present invention, the population of EVs according to the invention is loaded with exogenous molecules by electroporation.

The inventors have surprisingly found that a reduction of the number of pulses used in electroporation while decreasing the efficiency of loading an exogenous molecule into an extracellular vesicle, is also accompanied by a decreased damage to the extracellular vesicle and thereby improves integrity. It has been found that for extracellular vesicles carrying an endogenous cargo having a biologic activity the negative impact on the endogenous biologic activity can be kept to a minimum using such a low damaging protocol. At the same time the overall biologic effect of the endogenous biologic activity and the biologic activity conferred by the exogenous molecules may be increased.

Accordingly, in one preferred embodiment of the present invention the composition of the present invention is obtainable by electroporation.

Preferably, the electroporation procedure is carried out at a voltage comprised between 500 and 900 Volt, with a number of pulses comprised between 1 and 10, the duration of each pulse being comprised between 10 and 25 milliseconds.

In a more preferred embodiment of the invention, the voltage of electroporation is comprised between 600 and 800 Volt, preferably between 700 and 800 Volt.

According to another preferred embodiment of the invention, the duration of the electroporation pulse is comprised between 18 and 22 milliseconds.

A preferred number of electroporation pulses is 2 to 4, more preferably 2.

Within the scope of the present invention is also a method of loading a population of extracellular vesicles (EVs) with an exogenous molecule, which comprises the step of subjecting the EVs in the population to electroporation, wherein the electroporation is carried out at a voltage comprised between 500 and 900 Volt, with a number of pulses comprised between 1 and 10, the duration of each pulse being comprised between 10 and 25 milliseconds.

The following experimental section is provided purely by way of illustration and is not intended to limit the scope of the invention as defined in the appended claims. In the following experimental section reference is made to the appended drawings, wherein:

FIG. 1 shows the effects of electroporation on the diameter size in a population of plasma EVs. EVs size was assessed by Nanosight nanoparticle tracking system after electroporation under the indicated conditions (voltage and number of pulses) or incubation in the presence of miRNA cel-miR-39-3p. (A) Graph illustrating the results of the analysis of mean and mode vesicle diameter size. (B) Graph representing the distribution of EVs diameter size as percentage relative to unloaded controls. The spread of diameter size within the different EVs populations was evaluated by determining the maximum size in different percentages of the EVs population: D10, 10% of EVs population, D50, 50% of EVs population, D90, 90% of EVs population. *p<0.05; **p<0.01. Abbreviation: EV, EVs derived from Plasma; EV incubated+cel-39, EVs after incubation with synthetic cel-miR-39-3p; 500V 1p, EVs after electroporation with cel-miR-39-3p using 500 Volts and 1 pulse of 20 ms; 500V 10p, EVs after electroporation with cel-miR-39-3p using 500 Volts and 10 pulses of 20 ms; 750V 1p, EVs after electroporation with cel-miR-39-3p using 750 Volts and 1 pulse of 20 ms; 750V 10p, EVs after electroporation with cel-miR-39-3p using 750 Volts and 10 pulses of 20 ms; 1000V 1p, EVs after electroporation with cel-miR-39-3p using 1000 Volts and 1 pulse of 20 ms; 1000V 10p, EVs after electroporation with cel-miR-39-3p using 1000 Volts and 10 pulses of 20 ms.

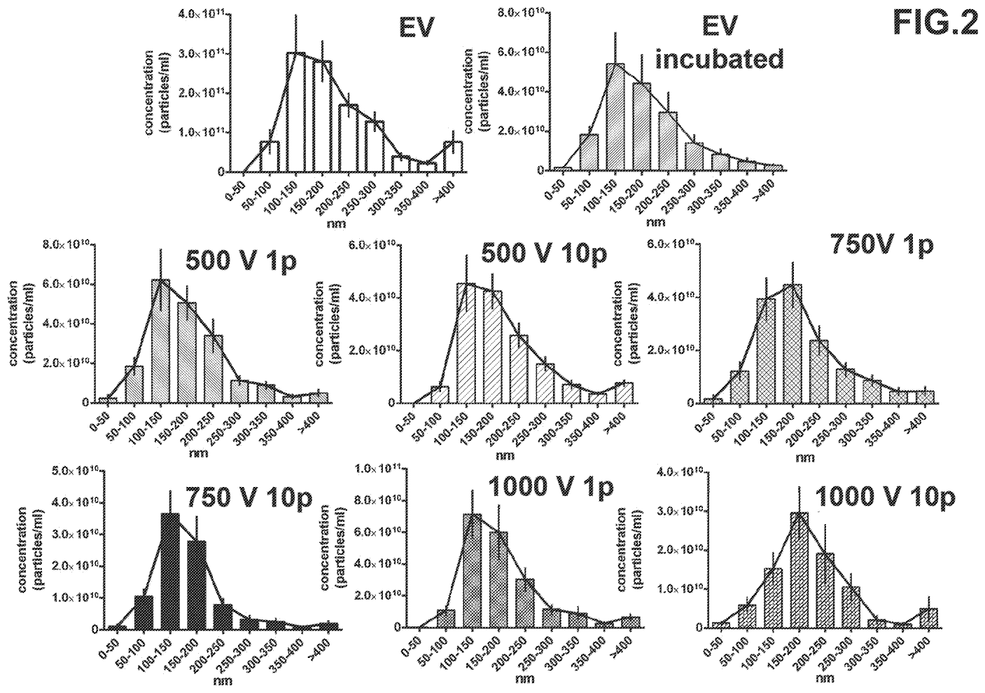

FIG. 2 shows the distribution profiles of the diameter size assessed by NTA in plasma-derived EVs populations after electroporation under the indicated conditions (voltage and number of pulses) or incubation in the presence of miRNA cel-miR-39-3p (n=3). As control, EVs populations were used which were not exposed to electrical pulse or incubation. Data±SEM.

FIG. 3 shows the loading efficiency and RNA content in a population of plasma-derived EVs after electroporation under the indicated conditions (voltage and number of pulses) or incubation in the presence of miRNA cel-miR-39-3p. (A) Graph representing EVs total RNA content (RNA nanogram in 10⁹ EVs) relative to control EVs (unloaded EVs, n=3). (B) Graph representing relative quantification (RQ) of miRNA expression in the examined EVs populations, measured by qRT-PCR. RQ values were determined using the endogenous RNU6B as housekeeping control, and normalized to control EVs (unloaded EVs, n=3). (C) Graph representing relative quantification of miRNA uptake, determined by qRT-PCR, in TEC target cells treated for 24 hours with 30,000 EVs/cell. Data were normalized to RNU6B housekeeping control (n=4). *p<0.05; **p<0.01; ****p<0.001. Abbreviation: EV, EVs derived from Plasma; EV incubated+cel-39, EVs incubated with synthetic cel-miR-39-3p; 500V 1p, EVs after electroporation with cel-miR-39-3p using 500 Volts and 1 pulse of 20 ms; 500V 10p, EVs after electroporation with cel-miR-39-3p using 500 Volts and 10 pulses of 20 ms; 750V 1p, EVs after electroporation with cel-miR-39-3p using 750 Volts and 1 pulse of 20 ms; 750V 10p, EVs after electroporation with cel-miR-39-3p using 750 Volts and 10 pulses of 20 ms. Data±SEM.

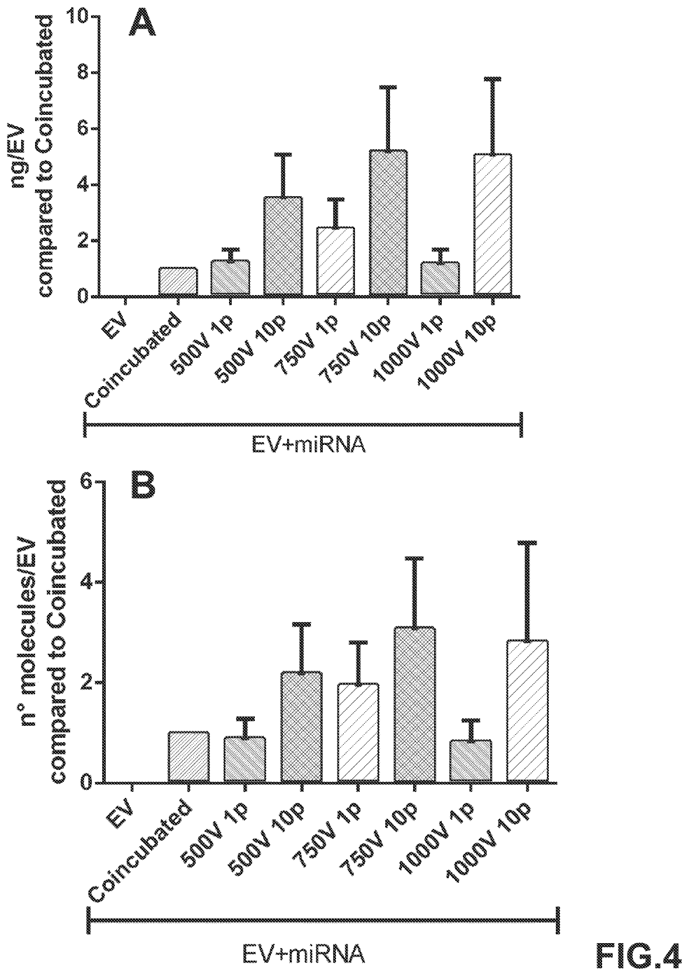

FIG. 4 shows the quantification of the amount of exogenous miRNA loaded in plasma-derived EVs following electroporation, measured as nanograms (A) or molecules number (B) (n=3). Data are normalized versus the results of EVs co-incubation with miRNA. Abbreviation: EV, unloaded EVs; co-incubated, EVs co-incubated with synthetic cel-miR-39-3p; 500V 1p, EVs after electroporation with cel-miR-39-3p using 500 Volts and 1 pulse of 20 ms; 500V 10p, EVs after electroporation with cel-miR-39-3p using 500 Volts and 10 pulses of 20 ms; 750V 1p, EVs after electroporation with cel-miR-39-3p using 750 Volts and 1 pulse of 20 ms; 750V 10p, EVs after electroporation with cel-miR-39-3p using 750 Volts and 10 pulses of 20 ms. Data±SEM.

FIG. 5 shows the effect of electroporation on original endogenous RNA and miRNA content in EVs populations. (A) Graph illustrating spectrophotometric quantification of total RNA content in unloaded EVs and in EVs after electroporation in the presence or absence of miRNA cel-miR-39-3p. Data are expressed as nanogram of total RNA in 10⁹ EVs relative to unloaded control EVs (n=6). (B) Graph illustrating the expression levels of a panel of miRNAs determined by qRT-PCR in unloaded EVs, and in EVs after electroporation in the presence or absence of miRNA cel-miR-39-3p. The miRNA relative expression is represented as RQ values normalized to global miRNA expression in each sample (n=4). (C) Heatmap representation of miRNAs expression levels (RQ values) for each sample, using average linkage as clustering method and Euclidean distance measurement. Abbreviation: EV, unloaded EVs; EV electroporated, EVs electroporated in the absence of cel-miR-39-3p; EV electroporated+cel-39, EVs electroporated in the presence of cel-miR-39-3p. Data±SEM. ****p<0.001 versus EV CTR.

FIG. 6 shows the effect of electroporation on total protein content, classical vesicular marker cargo and surface markers composition in EVs populations. (A) Graph illustrating the total protein content measured in unloaded EVs and in EVs after electroporation in the presence or absence of miRNA cel-miR-39-3p. Data are expressed as microgram (μg) of total protein in 10⁹ EVs (n=4). The comparison between electroporated EVs and unloaded controls did not reveal any statistically significant difference (ns=p-value>0.05). (B) Graph illustrating the results of FACS analysis of surface protein markers in unloaded EVs and in EVs after electroporation in the presence or absence of miRNA cel-miR-39-3p. Data are expressed as percentage of fluorescent signal intensity (n=4). (C) Western blot analysis of a set of classical markers (CD29, CD63, TSG101, CD81, CD9) in EVs following electroporation. Representative blot image (on the left); bar graph (on the right) illustrating vesicular markers expression in EVs after electroporation in the presence or absence of miRNA cel-miR-39-3p, relative to unloaded controls (EV). Protein expression was normalized on the total protein loaded and compared to control EVs (n=3). Abbreviation: EV, unloaded EVs; EV electroporated, EVs electroporated in the absence of cel-miR-39-3p; EV electroporated+cel-39, EVs electroporated in the presence of cel-miR-39-3p. Data±SEM.

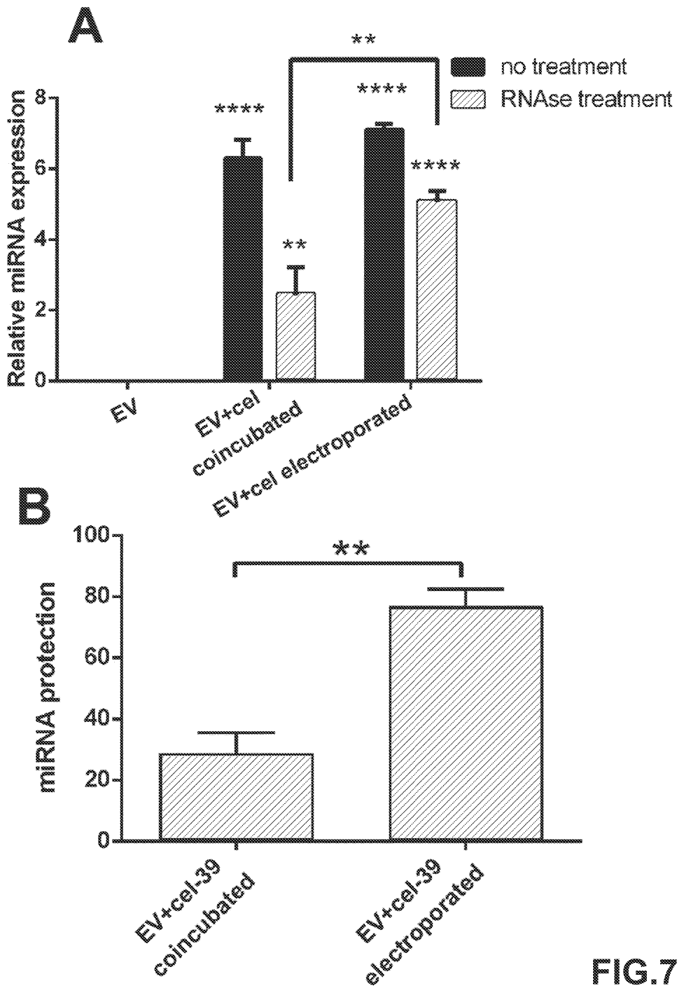

FIG. 7 shows the effects of RNAse treatment on the level of miRNA cel-miR-39-3p in EVs loaded with this synthetic miRNA by electroporation (EV+cel electroporated) or co-incubation (EV+cel co-incubated). A comparative analysis was carried out between EVs treated for 30 minutes with RNAse (0.2 μg/ml) and untreated EVs. (A) Graph representing miRNA cel-miR-39-3p expression measured as ln(RQ) values by qRT-PCR, relative to unloaded EVs. As housekeeping control, it was used RNU6B (n=4). (B) Graph representing the percentage of miRNA cel-miR-39-3p protected from RNAse in EVs loaded with this synthetic miRNA by co-incubation or electroporation. Data were calculated by comparing the ΔCt of the miRNA determined by qRT-PCR in the RNAse treated EVs with the ΔCt measured in not treated EVs (untreated controls were considered as 100%) (n=3). Data±SEM. **p<0.01, ****<0.001

FIG. 8 shows the pro-apoptotic effect on HepG2 cells of plasma EVs electroporated with anti-tumor miRNAs. HepG2 cells (30,000 EVs/cell) were treated for 24 hours with plasma EVs loaded with anti-tumor miRNAs hsa-miR-451a and hsa-miR-31-5p by electroporation and the effects on cell apoptosis or cellular gene expression were evaluated in comparison with control cells. (A) Graph representing the ratio of cancer cell apoptosis relative to untreated cells after stimulation with EVs loaded with different doses of miRNAs (n=6). (B) and (C) Graphs illustrating the expression levels of the HepG2 genes target of hsa-miR-31-5p (B) and hsa-miR-451a (C), respectively, compared to control samples (n=6). (D) Graph illustrating the results of the apoptosis assay on cancer cells treated with EVs loaded with dose×1 miRNA by electroporation or co-incubation, and treated with RNAse (n=5). Apoptosis of HepG2 cells was evaluated by using the Annexin kit Muse as the percentage of apoptotic cancer cells after treatment with the different EV samples, compared to controls (CTR−). Data±SEM. *p<0.05, **p<0.01, ***p<0.005, ****p<0.001. Abbreviation: CTR, cells cultured in DMEM 0% FCS; CTR+, cells treated with doxorubicin (150 ng/ml); EV, control EVs; EVi, EVs incubated; Eve, EVs electroporated.

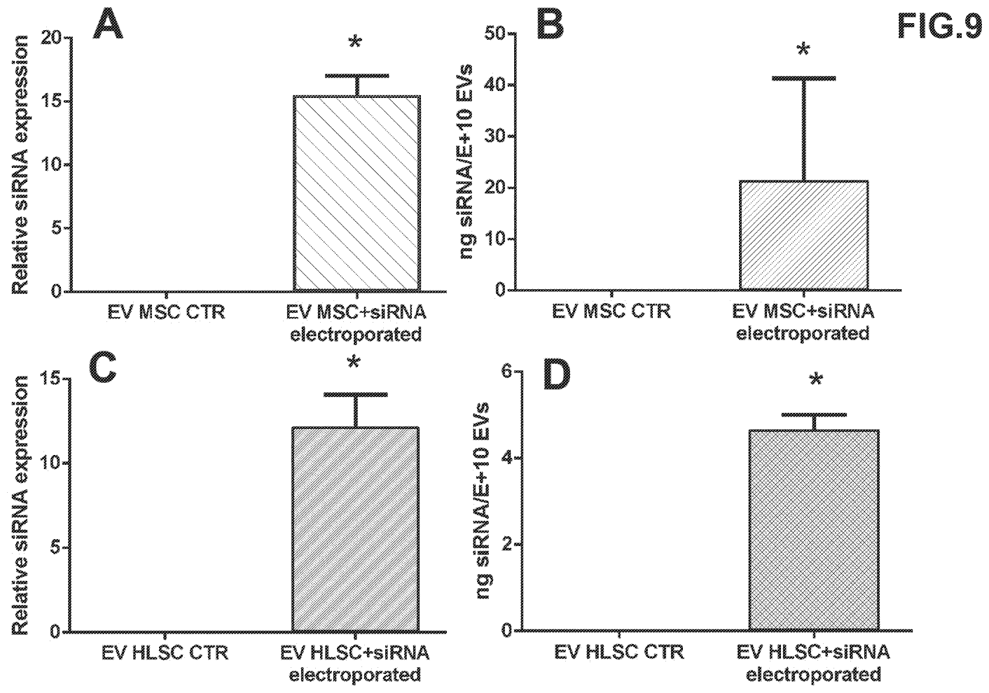

FIG. 9 shows validation of active loading of siRNA PCS-C2 into adult stem cell EVs by electroporation. siRNA PCS-C2 expression levels were determined in MSC-EVs (A) and HLSC-EVs (C) by qRT-PCR using the −ΔΔCt method, employing endogenous RNU6B as housekeeping control and normalized to unloaded EVs (EV CTR) (n=4). Loading of siRNA into EVs was also quantified as nanograms/10¹⁰ EVs in the MSC-EVs (B) and HLSC-EVs (D) populations (n=3). Data±SEM. *p<0.05.

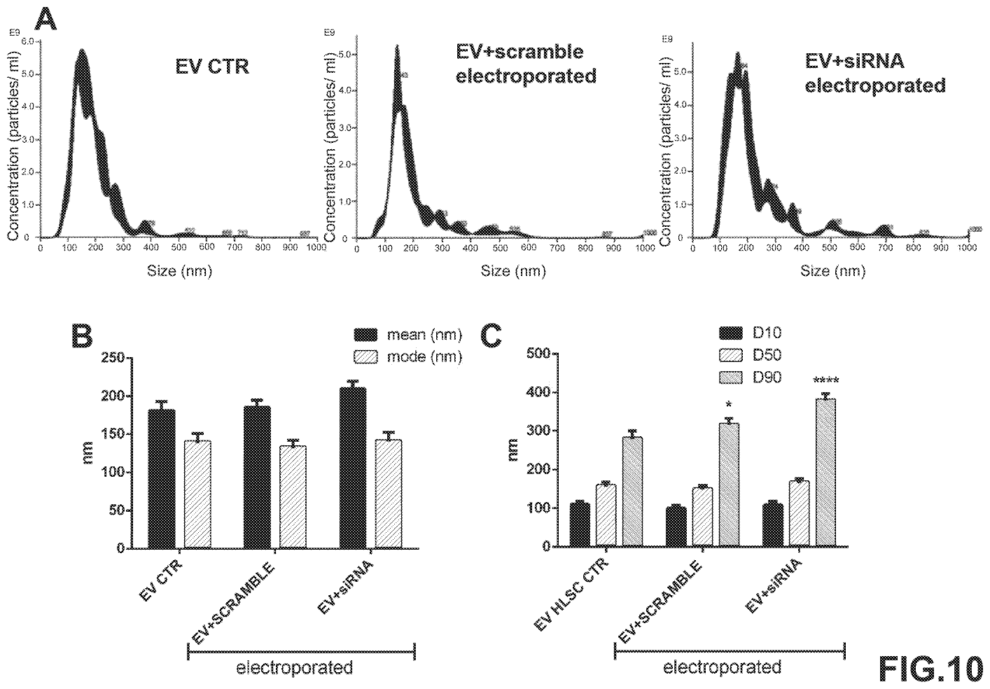

FIG. 10 shows the effects of electroporation at 750 V with 10 pulses of 20 ms on the diameter size in a population of HLSC-EVs. (A) Representative images of NTA profiles of control HLSC-EVs and HLSC-EVs after electroporation in the presence of siRNA PCS-C2 or scramble siRNA (B) Graph illustrating the results of the analysis of mean and mode vesicle diameter size. (C) Graph representing the distribution of EVs diameter size expressed as nanometer (nm), compared to unloaded controls. The spread of diameter size within the different EVs populations was evaluated by determining the maximum size in different percentages of the EVs population: D10, 10% of EVs population, D50, 50% of EVs population, D90, 90% of EVs population.*p<0.05; ****p<0.001. Data±SEM. Abbreviation: EV CTR, unloaded HLSC-EVs; EV+scramble electroporated, HLSC-EVs after electroporation with scrambled siRNA sequence; EV+siRNA electroporated, HLSC-EVs after electroporation with siRNA PCS-C2

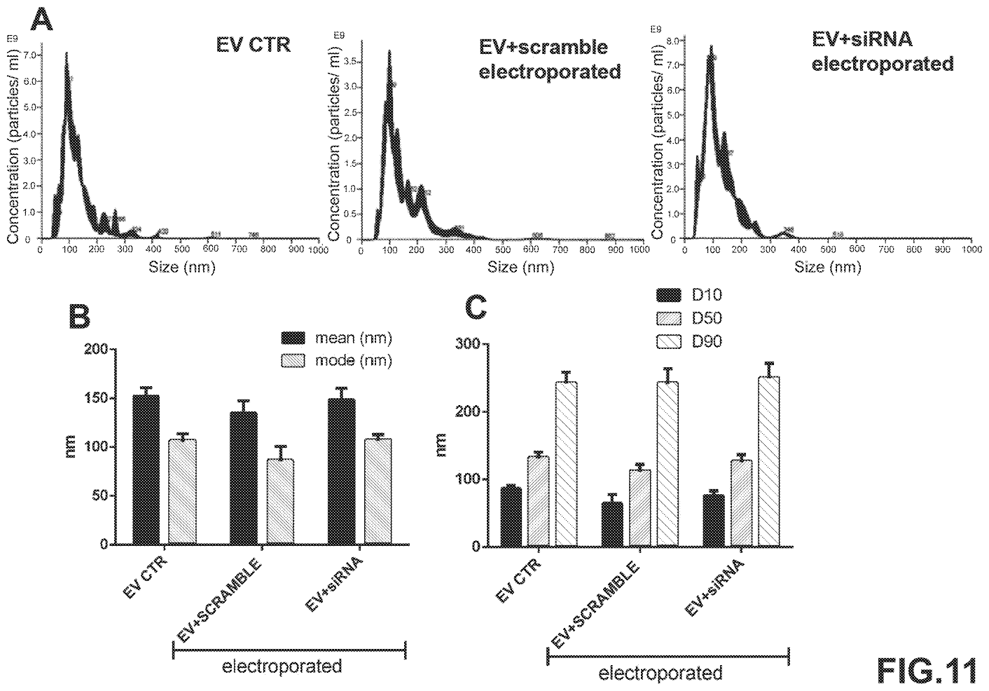

FIG. 11 shows the effects of electroporation on the diameter size in a population of MSC-EVs. (A) Representative images of NTA profiles of control MSC-EVs and MSC-EVs after electroporation in the presence of siRNA PCS-C2 or scrambled siRNA sequence. (B) Graph illustrating the results of the analysis of mean and mode vesicle diameter size. (C) Graph representing the distribution of EVs diameter size expressed as nanometer (nm), compared to unloaded controls. The spread of diameter size within the different EVs populations was evaluated by determining the maximum size in different percentages of the EVs population: D10, 10% of EVs population, D50, 50% of EVs population, D90, 90% of EVs population.* p<0.05; ****p<0.001. Data±SEM. Abbreviation: EV CTR, unloaded MSC-EVs; EV+scramble electroporated, MSC-EVs after electroporation with scrambled siRNA sequence; EV+siRNA electroporated, MSC-EVs after electroporation with siRNA PCS-C2.

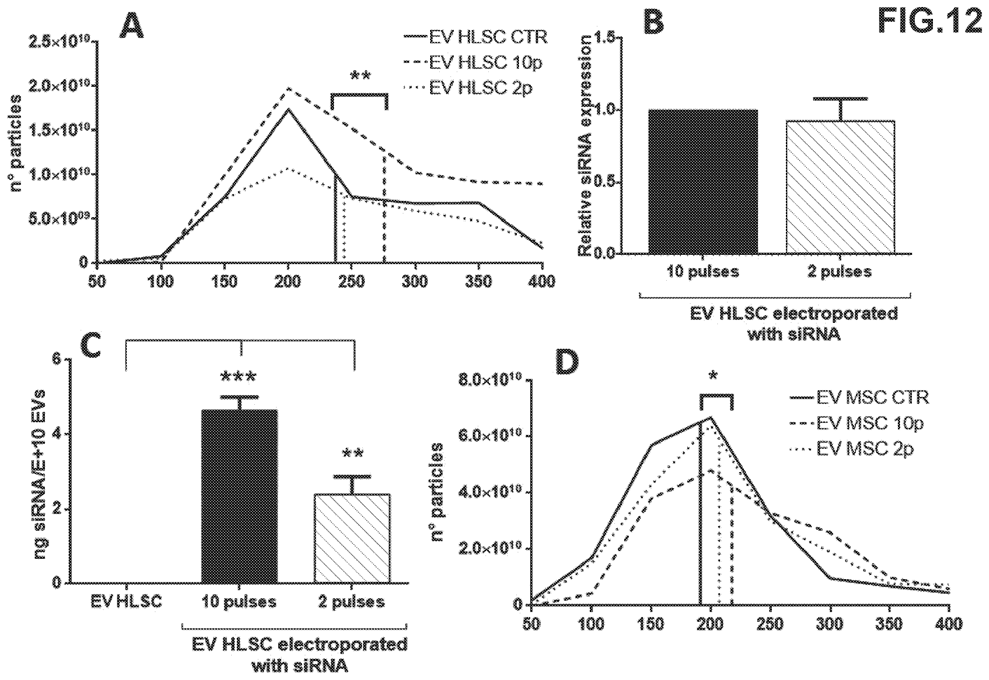

FIG. 12 shows the effects of different electroporation conditions on integrity and loading efficiency in populations of adult stem cell EVs. HLSC- and MSC-EVs were loaded with siRNA PCS-C2 by electroporation at 750 Volt with 10 or 2 pulses of 20 ms, and data compared to unloaded EVs as control. (A) HLSC-EVs and (D) MSC-EVs diameter size distribution profiles assessed by NTA (n=3). Each line represents data from a single electroporation condition (750 V with 10 pulses of 20 ms; 750 V with 2 pulses of 20 ms). Mean values (nm) are indicated by vertical lines. Active siRNA loading into HLSC-EVs is reported as relative expression level (RQ value) determined by qRT-PCR (B) using endogenous RNU6B as housekeeping control, and quantified as nanograms/10¹⁰ EVs (C), compared to unloaded EVs as control (n=3). Data±SEM. *p<0.05 **p<0.01, ***p<0.001.

FIG. 13 shows functional evaluation of adult stem cell EVs after electroporation. MSC-EVs (n=18) and HLSC-EVs (n=12) were loaded with siRNA PCS-C2 using different electroporation conditions (750 Volt with 10 or 2 pulses of 20 ms). The maintenance of pro-angiogenic activity of siRNA-loaded EVs was evaluated by using the tubulogenesis assay on endothelial cells, and compared to control EVs. Target cells were seeded 25,000 cells/well and the length of vessels was measured after 24 hours of treatment with EVs (50,000 EVs/cell). (A) and (B) In the upper panels, representative micrographs showing vessels formation. In the lower panels, bar graphs representing the levels of vessels formation determined in all samples relative to untreated endothelial cells (CTL−) along with the comparison of vessel formation activity between loaded adult stem cell EVs and unloaded EVs (horizontal lines). Data±SEM. **p<0.005; ****p<0.001; ns, not significant p>0.05. Abbreviation: CTL−, endothelial cells cultured in DMEM plus 5% EVs-depleted fetal calf serum; CTL+, endothelial cells cultured in EndoGRO medium; EV MSC, unloaded MSC-EVs; EV MSC 10p, MSC-EVs after electroporation with siRNA PCS-C2 using 750 Volts and 10 pulses of 20 ms; EV MSC 2p, MSC-EVs after electroporation with siRNA PCS-C2 using 750 Volts and 2 pulses of 20 ms EV HLSC, unloaded HLSC-EVs; EV HLSC 10p, HLSC-EVs after electroporation with siRNA PCS-C2 using 750 Volts and 10 pulses of 20 ms; EV HLSC 2p, HLSC-EVs after electroporation with siRNA PCS-C2 using 750 Volts and 2 pulses of 20 ms.

### 1. MATERIAL AND METHODS

### 1.1 Cell Culture

Human tumoral endothelial cell line TEC was established and maintained in culture in Endogro basal complete medium (Merck Millipore, Burlington, Mass., USA). Briefly, TECs were isolated from renal clear-cell carcinomas and previously characterized as endothelial cells by morphology, positive staining for vWF antigen, CD105, CD146, and vascular endothelial-cadherin and negative staining for cytokeratin and desmin

Human hepatoma cell line HepG2 (American Type Culture Collection, Manassas, Va., USA) was cultured in Dulbecco's modified Eagle's medium (DMEM) containing 10% fetal bovine serum (FBS).

Human microvascular endothelial cell line HMEC (American Type Culture Collection, Manassas, Va., USA) was cultured in Endogro basal complete medium (Merck Millipore, Burlington, Mass., USA) and 10% fetal calf serum (FCS).

Bone marrow MSCs were purchased by Lonza (Basel, Switzerland). Cells were used up to the seventh passage of culture. MSCs characterization was performed by cytofluorimetric analysis for the expression of the typical mesenchymal markers CD29, CD73, CD44, α4- and α5 integrins.

HLSCs were isolated from human cryopreserved normal adult hepatocytes (Lonza, Basel, Switzerland). Briefly, hepatocytes were first cultivated for 2 weeks in Hepatozyme-SFM medium (Gibco, Grand Island, N.Y., USA), then in α-MEM/EBM-1 media (3:1) (Invitrogen, Carlsbad, Calif., USA) added with HEPES (12 mM, pH 7.4), L-glutamine (5 mM) penicillin (50 IU/ml), streptomycin (50 μg/ml) (all from Sigma, St. Louis, Mo., USA), and fetal calf serum (FCS) (10%) (Invitrogen). The cells were expanded and characterized. The characterization of HLSCs by cytofluorimetric analysis demonstrated the expression of the mesenchymal stem cell markers but not of the endothelial and hematopoietic markers. HLSCs also expressed α-fetoprotein, human albumin, vimentin and nestin resident stem cell markers, but not CD34, CD117 and cytokeratin 19 oval cell markers. In addition, HLSCs were positive for the Nanog, Sox2, Oct4 and SSEA4 embryonic stem cell markers. HLSCs were shown to undergo osteogenic, endothelial and hepatic differentiation under appropriate culture conditions. Cells were used up to the seventh passage of culture. within the seven passages.

### 1.2 Extracellular Vesicle Isolation

Plasma-derived EVs were isolated from frozen human plasma of healthy blood donors provided by the Blood Bank of “Citta della Salute e della Scienza di Torino”. All samples were obtained after informed consent and approval by the internal Review Board of the Blood Bank. EVs from each donor were isolated from 250 ml plasma bags. Briefly, plasma samples were centrifuged at 1,500 g for 20 minutes to remove debris and apoptotic bodies. The supernatant was subsequently ultracentrifuged at 100,000 g for 2 hours at 4° C. using a 70 ml polycarbonate tube (SW 45 Ti rotor, Beckman Coulter Optima L-90 K ultracentrifuge, Indianapolis, Ind.). Samples were then washed with saline buffer solution and ultracentrifuged at 100,000 g for 2 hours at 4° C.

In order to isolate adult stem cell EVs, HLSC and MSC cells were cultured in the presence of their expansion medium until 80% of confluence. EVs were isolated from the supernatants of HLSC and MSC cells cultured overnight in Dulbecco's modified Eagle's medium (DMEM) using first differential centrifugation (1,500 g for 20 minutes to remove debris and apoptotic bodies) and then ultracentrifugation at 100,000 g for 2 hours at 4° C. in a 70 ml polycarbonate tube (SW 45 Ti rotor, Beckman Coulter Optima L-90 K ultracentrifuge, Indianapolis, Ind.). EVs pellets from plasma and cultured cells were then resuspended in saline buffer solution with 1% of DMSO, filtered through 0.22 micrometer filters to sterilize and stored at −80° C. EVs aliquots were then thawed and used for biological assays and molecular analysis.

### 1.3 EVs Analysis by NanoSight

EVs size and concentration were analyzed by nanoparticle tracking analysis (NTA), using the NanoSight LM10 system (NanoSight Ltd., Amesbury, UK), equipped with a 405 nm laser and with the NTA 3.1 analytic software). The Brownian movements of the EVs present in the sample subjected to a laser light source were recorded by a camera and converted into size and concentration parameters by the NTA software using the Stokes-Einstein equation. For all acquisition, camera levels were set at 16 and three videos of 30 s duration were recorded for each sample. Briefly, EVs were diluted (1: 1000 plasma-derived EVs and 1: 200 adult stem cell-derived EVs and nucleic acid-loaded EVs) in 1 ml vesicle-free saline solution (Fresenius Kabi, Runcorn, UK). NTA post-acquisition settings were optimized and maintained constant across all samples, and each video was then analyzed to calculate the concentration of EVs in the population under analysis along with the mean and mode vesicle diameter size and the different size distributions (D10, D50-median- and D90). D10=10% of vesicles have a diameter below the size indicated as D10; D50=50% of vesicles have a diameter below the size indicated as D50; D90=90% of vesicles have a diameter below the size indicated as D90.

### 1.4 FACS Characterization of EVs

Plasma-derived EVs were also characterized by cytofluorimetric analysis using the CytoFLEX flow cytometer (Beckman Coulter, Indianapolis, Ind.) together with the CytExpert software. The following FITC (fluorescein isothiocyanate) or APC (allophycocyanin) conjugated antibodies were used: anti-CD9, -CD19, -CD81, -CD86, -CD90, -HLA DR, -CD47, -CD34 (BD Biosciences, San Jose, Calif., USA), anti-CD40, -CD31, -CD144, -CD3, -CD146, -CD105 (Miltenyi Biotec, Bergisch Gladbach, Germany), anti-CD5 (Thermo Fisher Scientific, Waltham, Mass., USA) and anti-HLA ABC (BioLegend, San Diego, Calif., USA). Conjugated mouse non-immune isotypic immunoglobulin G (IgG) (Miltenyi Biotec, Bergisch Gladbach, Germany) was used as control. Briefly, EVs samples (5×10⁸ particles) diluted 1:3, were labeled with the above-listed conjugated antibodies for 15 minutes at 4° C. and, immediately after labelling, samples were acquired.

### 1.5 EVs Loading

Loading of EVs populations was performed using electroporation on a Neon Transfection System (Thermo Fisher Scientific, Waltham, Mass., USA) following manufacturer's instructions. For loading experiments with plasma EVs, the miRNAs hsa-miR-451a (SEQ ID NO. 1), hsa-miR-31-5p (SEQ ID NO. 3) and cel-miR-39-3p (SEQ ID NO. 5: 5′ UCACCGGGUGUAAAUCAGCUUG 3′) were used. Briefly, plasma EVs and miRNAs molecules (Qiagen, Hilden, Germany) were mixed (3×10¹⁰ EVs and 10 pmol, dose×1, 5 pmol, dose×½, 20 pmol, dose×2 miRNA) and diluted in the electroporation buffer R (Thermo Fisher Scientific, Waltham, Mass., USA) to a final volume of 100 μl. The mixture was subjected to electroporation using a pulse width of 20 milliseconds (ms) at increasing voltages (500, 750, 1000 V) with increasing number of pulses (from 1 to 10). Following electroporation, the mixture was incubated for 30 minutes at 37° C. and overnight at 4° C. EVs samples after co-incubation with miRNAs or EVs samples after electroporation in the absence of miRNAs were used as controls.

For the electroporation experiments with adult stem cell EVs, the following siRNA molecules were used: PCS-C2 (SEQ ID NO. 6: 5′ AGGUGUAUCUCCUAGACACTT 3′, sense strand; SEQ ID NO. 7: 5′ GUGUCUAGGAGAUACACCUTT 3′, antisense strand) and Scramble siRNA (SEQ ID NO. 8: 5′ GAGAUUACGAUUGCUGGGCTT 3′, sense strand; SEQ ID NO. 9: 5′ GCCCAGCAAUCGUAAUCUCTT 3′, antisense strand). A total of 3×10¹⁰ adult stem cell EVs were mixed with 10 pmol siRNA, either PCS-C2 or Scramble, and diluted in the electroporation buffer to a final volume of 100 μl. The mixture was electroporated using a pulse width of 20 ms, at 750 Volt with a different number of pulses, 2 or 10. Following electroporation, the mixture was incubated for 30 minutes at 37° C. and overnight at 4° C.

After electroporation of plasma EVs or adult stem cell EVs, in order to eliminate free not bound miRNA or siRNA molecules, the electroporated samples were washed by ultracentrifugation at 100,000 g for 2 hours at 4° C. using a 10 ml polycarbonate tube (SW 90 Ti rotor, Beckman Coulter Optima L-90 K ultracentrifuge, Indianapolis, Ind.). Finally, EVs pellets were resuspended in saline buffer solution with 1% of DMSO, analyzed by Nanosight and stored at −80° C. for downstream analysis. In these experiments, samples of adult stem cell EVs electroporated with the scrambled siRNA sequence or samples of adult stem cell EVs electroporated in the absence of siRNA were used as controls.

### 1.6 RNAse Treatment

To test the ability of EVs to protect their cargo from microenvironmental degradation, the samples of loaded EVs were treated with RNAse A (Thermo Fisher Scientific, Waltham, Mass., USA), using a concentration of 0.2 μg/ml, for 30 minutes at 37° C. Following the incubation period, a RNAse inhibitor (Thermo Fisher Scientific, Waltham, Mass., USA) was added to the mixture to stop the reaction according to manufacturer's protocol, and RNase-treated EVs were washed by ultracentrifugation at 100,000 g for 2 hours at 4° C. using a 10 ml polycarbonate tube (SW 90 Ti rotor, Beckman Coulter Optima L-90 K ultracentrifuge, Indianapolis, Ind.). Eventually, EV pellets were resuspended in saline buffer solution with 1% of DMSO and stored at −80° C. for downstream analysis.

1.7 RNA Isolation and qRT-PCR

Total RNA was isolated from the EVs populations using the miRNeasy Mini Kit (Qiagen, Hilden, Germany) according to the manufacturer's instruction, and quantified using spectrophotometric analysis. Absorbance (A) values at 260 nm and 280 nm were measured with a VWR mySPEC spectrophotometer (VWR, Radnor, Pa., USA). An OD of 1 at 260 nm was equated to 40 μg/ml RNA. The A260/A280 ratio was used to determine the RNA purity of the samples. A pure RNA sample has an A260/A280 ratio of 1.8-2.0

The composition of small RNAs in EVs populations was assessed by capillary electrophoresis on an Agilent 2100 Bioanalyzer using the small RNAs kit (Agilent Technologies, Inc., Santa Clara, Calif.).

The expression levels of mRNAs and miRNAs were analyzed by performing quantitative reverse transcriptase polymerase chain reaction (qRT-PCR) in triplicate using a 96-well QuantStudio 12K Flex Real-Time PCR (qRT-PCR) system (Thermo Fisher Scientific, Waltham, Mass., USA).

Briefly, to assess gene-downregulation in HepG2 cells in the apoptosis assay, cDNA was generated by reverse-transcription on cellular RNA samples using the “High-Capacity cDNA Reverse Transcription Kit” (Applied Biosystems, Foster City, Calif., USA). Five nanograms of cDNA were then combined to the “SYBR GREEN PCR Master Mix” (Applied Biosystems, Foster City, Calif., USA) according to manufacturer's instruction, and the GAPDH gene was used as housekeeping control.

For miRNAs expression analysis, the “miScript SYBR Green PCR Kit” (Qiagen, Hilden, Germany) was used. Briefly, the samples of miRNAs were reverse transcribed into cDNA using the “miScript Reverse Transcription Kit” (Qiagen, Hilden, Germany). The qRT-PCR experiments were carried out using 3 ng of cDNA in each reaction as described by the manufacturer's protocol (Qiagen, Hilden, Germany). The RNU6B small nucleolar RNA was used as control.

The levels of mRNA and miRNA were compared across samples based on relative expression data normalized using appropriate endogenous controls. The real-time PCR data were analyzed using the ΔΔCt method, and/or the fold changes in expression levels (RQ=2^{-ΔΔCt}) were calculated for all EVs samples, compared to controls. In details, ΔCt was measured as Ct difference between miRNA/mRNA of interest and housekeeping control. ΔΔCt was calculated as ΔCt difference between sample and control. RQ was the calculated as 2\hat{ }(−ΔΔCt).

In RNase-treated EVs samples, the percentage of protected miRNA was calculated based on cycle threshold (Ct) differences between treated and untreated EVs. More specifically, the ΔCt values measured for miRNAs in the RNAse treated samples were compared to the ΔCt values measured in untreated samples (untreated controls were considered as 100%). For the generation of standard curve to perform absolute quantification, miRNA cel-miR-39-3p and siRNA PCS-C2 were spectrophotometrically quantified (mySPEC, VWR, Radnor, Pa., USA) and 200 ng of RNA were reverse transcribed using the miScript Reverse Transcription Kit (Qiagen, Hilden, Germany). The cDNA thus generated was serially diluted 1:5 from an initial quantity of 2.4 ng to produce 10 dilutions. These serial dilutions were run in 5 replicates using Relative Standard Curve on 96-well QuantStudio 12K Flex Real-Time PCR (qRT-PCR) system according to the manufacturer's protocol (Thermo Fisher Scientific, Waltham, Mass., USA). The Standard curve was used to convert the cycle threshold (Ct) values measured for each sample into the corresponding number of microRNA or siRNA copies.

To analyze miRNA transfer from EVs to target cells, TEC or HepG2 were pre-plated in a 24-well plates and stimulated with 30,000 EVs/cell for 24 hours. Then, cell samples were subjected to RNA extraction and qRT-PCR analysis as described above

### 1.8 Protein Extraction and Western Blot Analysis

Proteins were extracted from EVs samples by using RIPA buffer (20 nM Tris-HCl, 150 nM NaCl, 1% deoxycholate, 0.1% sodium dodecyl sulfate, 1% Triton X-100, pH 7.8) supplemented with a cocktail of protease and phosphatase inhibitors (Sigma-Aldrich). The protein content of analyzed EVs was quantified by BCA Protein Assay Kit (Pierce, Thermo Fisher Scientific, Rockford, IL-61105, USA) following manufacturer's instruction. Briefly, 5 μl of EVs sample were dispensed into a 96-well plate and total protein concentrations was determined with a spectrophotometer using a linear standard curve established with bovine serum albumin (BSA). Thirty micrograms of proteins were separated by electrophoresis using a 7.5% gradient sodium dodecyl sulfate—polyacrylamide gel. The proteins were transferred to a PVDF membrane by the Trans-Blot® Turbo™ Transfer System (Bio-rad, Hercules, Calif., USA) and then immunoblotted with the following antibodies: anti-CD63 and anti-TSG101 (Santa Cruz Biotechnology, Dallas, Tex., USA), anti-CD81 and anti-CD9 (Abcam, Cambridge, UK) and anti-CD29 (Thermo Fisher Scientific, Waltham, Mass., USA). The protein bands were visualized using a ChemiDoc (Bio-rad, Hercules, Calif., USA) with an enhanced chemiluminescence detection kit (ECL) (GE Healthcare, Amersham, Buckinghamshire, UK). Protein quantification was performed normalizing sample amount to the total protein loaded detected by ponceau.

### 1.9 Apoptosis Assay

HepG2 cells were seeded at 25,000 cells/well into 24-well plates and cultured in serum free low-glucose DMEM in the absence (vehicle, CTR−) or presence of different populations of EVs (30,000 EVs/cell) for 24 hours. Cells maintained in low-glucose DMEM plus 150 ng/ml Doxorubicin were used as positive control (CTR+). Apoptosis was measured by using Muse™ Annexin V and Dead Cell Assay Kit (Merck Millipore, Burlington, Mass., USA) following the manufacturer's instructions. The assay is based on the detection of phosphatidylserine (PS) on the surface of apoptotic cells, using fluorescently labeled Annexin V in combination with the dead cell marker, 7-AAD. The results were shown as the percentage of apoptotic cells. compared to untreated cells.

### 1.10 Tubulogenesis Assay

The tubulogenesis assay is based on the in vitro formation of capillary-like structures on growth factor—reduced Matrigel (BD Bioscience, Franklin Lakes, N.J., USA). HMECs cells were seeded at 25,000 cells/well into 24-well plates in DMEM plus 5% EVs-depleted fetal calf serum (FCS) and stimulated with 50,000 EVs/cell for 24 hours. As negative controls, cultures of HMECs cells in DMEM plus 5% EVs-depleted FCS were used since under these growing conditions, endothelial cells do not exert angiogenetic activity. As positive controls, HMECs cells grown in the endothelial specific-EndoGRO basal medium (Merck Millipore, Burlington, Mass., USA) were used since such medium preserves their angiogenetic properties. Cell organization onto Matrigel was imaged with a Nikon Eclipse TE200. After incubation for 24 h, phase-contrast images (magnification, ×10) were recorded and the total length of the network structures was measured using ImageJ software. The total length per field was calculated in five random fields and expressed as a ratio to the respective control.

### 1.11 Statistical Analysis

Data were analyzed using the GraphPad Prism 6.0 Demo program. Statistical analyses were conducted using ANOVA with Dunnett's or Turkey's post-tests, or t-test where appropriated. The relative expression of miRNAs and mRNAs in samples was compared to suitable controls by Kruskal-Wallis ANOVA with Dunn's multiple comparisons test. Values were expressed as their mean and standard error of the mean (±SEM). Statistical significance was established at P<0.05 (illustrated as *p<0.05, **p<0.01, ***p<0.005, ****p<0.001).

### 2. RESULTS

2.1 Optimization of EVs Loading with an Exogenous Molecule

Human plasma from healthy donors is an easy and abundant source of EVs, with a recovery rate of about 5.33×10⁹ EVs (±2.40×10⁹)/ml of plasma (data not shown). In addition, the present inventors found that plasma-derived EVs were ineffective in their in vitro models, making them a useful source to test the potential therapeutic effects of specific miRNAs. EVs were isolated from human plasma of healthy donors by ultracentrifugation at 100,000 g for 2 h at 4° C. To define the most efficient electroporation protocol, the present inventors evaluated different electroporation parameters, including different voltages (500-, 750-, 1000 Volt) and different number of applied pulses (1, 2 or 10 pulses), each of 20 ms. To set up the electroporation (EP) protocol, the present inventors employed a synthetic miRNA (cel-miR-39-3p) which is easily detectable in human plasma-EVs and human cells because it is isolated from an unrelated organism (Caenhorabditis.elegans). Since electrical discharge of electroporation can damage EVs, the EV size distribution was taken into consideration when comparing the different protocols in order to select the most efficient and useful method of EVs loading. Results are shown in FIG. 1. In details, NTA analysis did not show any significant alteration in the mean and mode diameter size in EVs populations across all electroporation protocols compared to control EVs (FIG. 1A). However, data shown in FIG. 1B indicate an altered EV size distribution when EVs were subjected to electroporation with the Voltage 1000 Volt and 10 pulses. In fact, the present inventors found that when EVs were electroporated with the highest voltage and highest pulse number, the 10% of EV population showed a significantly increase of size in respect to control unloaded EVs, EVs subjected to co-incubation with miRNA cel-miR-39-3p or EVs subjected to electroporation with reduced voltages (500 V 1 pulse and 750 V 10 pulses).

The above-illustrated data reflect the shift in the size profile of EVs, i.e. the diameter size, after electroporation with higher voltages. In fact, the analysis of EVs subjected to electroporation with different protocols showed a similar EVs size distribution across the majority of samples, with a peak of EVs higher concentration around 100-150 nm. In particular, the profiles of unloaded EVs, EVs co-incubated and EVs subject to electroporation at 750V with 10 pulses of 20 ms were very similar. On the contrary, a relevant shift of the peak of EVs higher concentration to 150-200 nm was detected when applying the protocol employing 1000V with 10 pulses of 20 ms, suggesting that electroporation with higher voltages can damage EVs by inducing vesicles aggregation (FIG. 2).

The efficiency of electroporation was evaluated by measuring miRNA active loading into EVs along with the ability of EVs to transfer the loaded miRNA into recipient target cells. [mp1] The analysis of the total RNA content in electroporated EVs revealed a significant increase in the RNA content when EVs were subjected to high-voltage electroporation. In particular, the major RNA enrichment was achieved using 750 Volt with 10 pulses of 20 ms (FIG. 3A). The analysis of exogenous miRNAs encapsulated into EVs confirmed the achievement of higher miRNA enrichment with electroporation than using co-incubation. Electroporation at high voltages (750 and 1000 V) with 10 pulses of 20 ms resulted in more efficient EVs loading (FIG. 3B). Finally, the uptake of miRNA cel-39-3p [mp2] contained in loaded EVs into recipient target cells was evaluated, demonstrating that significant incorporation of synthetic miRNA occurred only when electroporation of EVs was carried out at 750 V or 1000 V with 10 pulses of 20 ms. Therefore, based on these results, the electroporation protocol using 750 Volt and 10 pulses of 20 ms was selected as the most suitable for EVs loading with an exogenous molecule and has been applied throughout the study of the present inventors.

To deeper investigate the efficiency of selected electroporation protocols, the present inventors calculated the amount (nanograms) and the number of molecules of exogenous miRNA loaded into EVs after electroporation, using a standard curve method. The graphs in FIG. 4 report miRNA nanograms (FIG. 4A) and miRNA number of molecules (FIG. 4B) which are detected in single EVs, normalized to EVs co-incubated with the same miRNA. Notably, the majority of electroporation protocols yielded increased enrichment of exogenous miRNAs hsa-miR-451a and hsa-miR-31-5p compared to co-incubation protocol, and electroporation carried out at 750 V with 10 pulses of 20 ms led to the highest loading of EVs, with an increase of miRNA content of at least 2 ng/10¹⁰ EVs. Table 1 shows the absolute number of exogenous miRNAs loaded into plasma EVs using the above-described approaches. Based on EVs integrity after electroporation and active loading of EVs, which enables exogenous miRNA transfer into recipient target cells, the present inventors selected the electroporation method at 750 V with 10 pulses of 20 ms as the most efficient and suitable for further experiments.

TABLE 1
Exogenous miRNA amount (number of molecules) loaded into EVs.

| | Mean | Std. Error |
|---|---|---|
| EV | 0.0 | 0.0 |
| EV + cel-39 Co-incubated | 15.6 | 12.2 |
| EV + cel-39 500 V 1 p | 10.3 | 6.8 |
| EV + cel-39 500 V 10 p | 12.6 | 2.7 |
| EV + cel-39 750 V 1 p | 12.6 | 3.9 |
| EV + cel-39 750 V 10 p | 24.9 | 11.5 |
| EV + cel-39 1000 V 1 p | 11.5 | 8.5 |
| EV + cel-39 1000 V 10 p | 23.1 | 11.8 |

### 2.2. Electroporation Effects on EVs

Permeabilization of EVs membrane caused by electroporation can lead to the loss of molecules contained in these vesicles, thereby altering their original cargo and their biological activity. To determine whether the electroporation method can modify the endogenous content of EVs, the present inventors analyzed the RNA, miRNAs and protein cargo in control EVs and in EVs after electroporation in the presence or absence of a miRNA. As expected, a significant increase in the RNA content was observed following electroporation with the miRNAs, whereas the quantification of EVs total RNA following electroporation in the absence of nucleic acid did not reveal any significant reduction in the RNA cargo compared to control EVs (p-value>0.05) (FIG. 5A). In addition, the present inventors analyzed the expression of a panel of miRNAs reported in the literature as highly expressed in EVs. A comparison was performed across the expression levels of these miRNAs measured by qRT-PCR in control EVs and in EVs after electroporation in the presence or absence of miRNA cel-miR-39-3p. The relative expression data thus measured demonstrated active EVs loading with miRNA cel-miR-39-3p compared to unloaded EVs and EVs electroporated in the absence of nucleic acid molecules. Notably, no significant variation was observed in the expression profile of analyzed miRNAs as determined in control EVs and in EVs subjected to electroporation in the presence or absence of miRNA cel-miR-39-3p (FIG. 5B). In fact, heatmap analysis revealed a similar expression pattern of all tested miRNAs in the EVs populations under examination (FIG. 5C), indicating that electroporation does not significantly modify the original cargo of endogenous miRNAs in EVs populations. Next, the present inventors evaluated the protein content in the EVs populations under analysis and found that electroporation in the presence or absence of the miRNA cel-miR-39-3p did not decrease the total amount of proteins packaged within the EVs (FIG. 6A). To further support protein content analysis, classical vesicular protein markers were evaluated, which are naturally enclosed within EVs. As shown in FIG. 6C, Western blot analysis of tetraspanins (CD63, CD81 and CD9), integrin 131 (CD29) and tumor susceptibility gene 101 (TSG101) confirmed the absence of alteration in the endogenous content of protein markers in EVs after electroporation, compared to unloaded EVs as control. To further evaluate if electroporation could qualitatively alter the composition of EV-membrane protein markers, FACS analysis was carried out on a panel of proteins reported as highly expressed in plasma EVs. The results of this analysis did not show any significant variation in the expression of surface protein markers in the EV populations electroporated in the presence or absence of miRNA cel-miR-39-3p compared to unloaded EVs (FIG. 6B). Briefly, the average reduction in expression level was determined as percentage for each surface marker analyzed in loaded EVs compared to unloaded controls, and a mean value of about 11.97% was calculated across these determinations as the maximum expression reduction in respect to controls. Overall, these results indicate that electroporation preserve EV membrane-protein composition.

### 2.3 EVs Protection of Loaded Exogenous Molecule

EVs are widely reported to protect their cargo from the microenvironmental degradation mediated by RNAse enzymes. To verify whether loaded exogenous molecules are actually encapsulated within the vesicles, EVs carrying the synthetic miRNA cel-miR-39-3p were analyzed by qRT-PCR after RNAse treatment. The present inventors investigated the resistance to RNAse displayed by EVs samples treated with a physiological dose of RNAse A, by comparing unloaded EVs and EVs electroporated or co-incubated with the specific miRNA (cel-miR-39-3p). Relative expression values of miRNA cel-miR-39-3p measured by qRT-PCR indicate that a significant enrichment of this miRNA into EVs was achieved after either electroporation or co-incubation (FIG. 7A). Notably, the RNAse treatment of EVs caused a reduction in the level of loaded miRNA cel-miR-39-3p. In particular, the present inventors observed a more evident degradation of the exogenous miRNA by using the co-incubation method. As shown in FIG. 7B, the protection against miRNA cel-miR-39-3p degradation achieved with co-incubation and electroporation correspond to 30% and 80%, respectively. These data suggest that electroporation enables a higher protection from environmental degradation than co-incubation.

2.4 Functional Evaluation of Plasma EVs Loaded with an Exogenous Molecule

Upon validation of EV integrity after electroporation, EVs were loaded with antitumor miRNAs and evaluated for their capacity to induce apoptosis in the human hepatocellular carcinoma cell line HepG2. For this purpose, the present inventors electroporated plasma EVs, which do not exhibit a natural pro-apoptotic activity in the employed in vitro model, with two synthetic miRNAs, namely hsa-miR-451a and hsa-miR-31-5p, which are known to promote apoptotic signals in HepG2 cells (Fonsato V. et al, Human liver stem cell-derived microvesicles inhibit hepatoma growth in SCID mice by delivering antitumor microRNAs. Stem Cells. 2012 September; 30(9):1985-98). Electroporation was carried out at 750 V with 10 pulses of 20 ms with different miRNA doses (the initial dose, ×1; half dose, ×1/2; double dose×2) to evaluate the biological effect of varying miRNA quantities. The apoptosis assay performed by treating HepG2 cells with loaded EVs as above described demonstrated a general significant increase in cancer cell apoptosis when EVs electroporated with hsa-miR-451a and hsa-miR-31-5p were used, compared to untreated cells (CTL−) (FIG. 8A). In particular, the miRNA initial dose (×1) was the most effective, suggesting that the additional increase in miRNA quantity did not potentiate the EV effect. On the contrary, the miRNA dose×½ was ineffective after both co-incubation and electroporation, suggesting that the miRNA loading was not sufficient to achieve a biological activity. A comparison between the two loading protocols using both miRNAs at higher dose revealed that EVs loaded by electroporation were more efficient in inducing cancer cell apoptosis than EVs loaded by co-incubation protocol. These data highlighted a more efficient miRNA enrichment of EVs and consequent biological activity following electroporation in comparison to co-incubation. To further validate the biological activity of exogenous molecule-loaded EVs, the present inventors evaluated the expression of genes target of hsa-miR-451a and hsa-miR-31-5p, which are involved in apoptotic or drug resistance pathways in recipient cells (FIGS. 8B and 8C). The treatment of cancer cells with EVs loaded with hsa-miR-31-5p by electroporation induced a significant down regulation of the target genes of this miRNA, compared to control cells, including CDK2, E2F2, SP1 and BCL2α genes (FIG. 8 B). Interestingly, EVs loaded with hsa-miR-31-5p by co-incubation did not induce any significant biological effect, thereby indicating the more effectiveness of the electroporation method. With regard to EVs loaded with hsa-miR-451a, the expression of BCL2α, CASP3, MDR1 and RAB14 genes was analyzed (FIG. 8C). By employing EVs loaded by co-incubation a significant reduction in expression level was detected only for the MDR1 gene, whereas a significant downregulation of the expression of BCL2α, MDR1 and RAB14 genes was achieved with EVs loaded with nucleic acid by electroporation. These data confirmed once again the higher efficacy of the electroporation procedure compared to co-incubation method. Finally, the present inventors investigated the ability of EVs loaded with hsa-miR-31-5p to maintain the anticancer effect after RNAse treatment. FIG. 8D shows that, after RNase treatment, only EVs loaded by electroporation were able to significantly promote cancer cell apoptosis after 24 hours of EVs stimuli. These data confirm a more efficient incorporation of miRNA in EVs following electroporation than co-incubation protocol.

### 2.5 Electroporation Effects of the Integrity of Adult Stem Cell EVs

To assess the viability of electroporation as a method for loading exogenous molecules into adult stem cells EVs, the present inventors applied the electroporation protocol at 750 Volt with 10 pulses of 20 ms to EVs isolated from MSCs and HLSCs in the presence of a synthetic siRNA (siRNA PCS-C2). By qRT-PCR analysis, a clear enrichment of the exogenous siRNA was demonstrated in the populations of adult stem cell EVs after electroporation (FIG. 9 A, C). The amount of siRNA PCS-C2 loaded in EVs was calculated as ng of siRNA loaded by 10¹⁰ EVs (FIG. 9 B,D).

Differences in EV membrane composition may depend on the cell origin. Thus, NTA analysis was performed to evaluate possible structural alterations of adult stem cell EVs after electroporation. In particular, the present inventors found that electroporated HLSC-EVs displayed NTA profiles and sizes similar to unloaded HLSC-EVs (FIG. 10 A, B). By contrast, the analysis of the diameter distribution in the HLSC-EVs populations revealed an increase in the maximum diameter size in the 90% of electroporated EVs population (D90) compared to unloaded controls. Such alteration may be ascribed to electroporation-induced EVs aggregation.

Exogenous molecule loading by electroporation did not induce any significant change in the mean, mode and distribution of diameter size in the population of MSCs-EVs (FIG. 11).

In order to avoid EVs damage, electroporation experiments with a lower number of pulses were conducted.

Loading of HLSC-EVs by electroporation using 2 pulses of 20 ms instead of 10 did not lead to increased EV mean size (FIG. 12A) and maximum size (D90, 90% of EV population) (FIG. 12B). Moreover, the relative enrichment of siRNA PCS-C2 measured in HLSC-EVs was similar when 2 or 10 pulses of 20 ms were used (FIG. 12C). Indeed, the electroporation method with 2 pulses achieved a significant siRNA PCS-C2 enrichment quantified as at least 2 ng/10¹⁰ EVs (FIG. 12D). Similar results were obtained when the electroporation protocol with 2 pulses was applied to load MSC-EVs with the siRNA PCS-C2 (FIG. 12 E,F). Overall, these data indicate that electroporation with a lower number of pulses (2 pulses of 20 ms) is a method suitable for loading exogenous molecules into adult stem cell EVs since this method preserves the integrity of the EVs in the population, thereby causing no damage, and at the same time enables efficient encapsulation of the exogenous molecule. Table 2 shows the increase as percentage of the mean diameter size in the populations of EVs subjected to electroporation with 2 or 10 pulses of 20 ms, compared to unloaded controls.

The maximum increase in diameter detected by the present inventors in the populations of adult stem cell EVs, which had previously been validated as not damaged by exogenous molecule loading, is 9.86%.

TABLE 2
Size alterations in adult stem cell EVs loaded with an exogenous molecule

| | Increase of mean |
|---|---|
| sample | size |
| EV HLSC CTR | 0.00% |
| EV HLSC 10 p | 12.11% |
| EV HLSC 2 p | 2.29% |
| EV MSC CTR | 0.00% |
| EV MSC 10 p | 18.74% |
| EV MSC 2 p | 9.86% |

2.6 Functional Evaluation of Adult Stem Cell EVs Loaded with an Exogenous Molecule

As a further assessment of the absence of damage in the population of adult stem cell EVs after active exogenous molecule loading, the present inventors verified whether these EVs maintain their biological activity compared to unloaded EVs. More particularly, the pro-angiogenic activity of unloaded control EVs and adult stem cell EVs subjected to electroporation at 750 Volt with 10 or 2 pulses of 20 ms, in the presence or absence of the siRNA PCS-C2, was analyzed carrying out a tubulogenesis assay on endothelial cells (FIG. 13). This assay measures the ability of endothelial cells, plated at subconfluent densities with the appropriate extracellular matrix support, to form capillary-like structures under tested stimuli. Vessel formation is quantified by measuring the number, length, or area of these capillary-like structures in two-dimensional microscope images. In Table 3 are summarized the results of the tubulogenesis analysis reported as percentage decrease in biological activity measured in electroporated adult stem cells EVs compared to unloaded controls. When ten electroporation pulses were used, both the MSC-EVs and HLSC-EVs populations showed decreased pro-angiogenic activity (biological activity reduction of 12.81% and 7.29%, respectively), compared to unloaded EVs (biological activity reduction of 0.00%). These results are in agreement with the presence of structural damage in EVs electroporated with a high number of pulses, as above illustrated. In contrast, as shown in the graphs of FIGS. 13A and B, the present inventors did not observe any significant decrease in biological activity compared to unloaded controls when the MSC-EVs and HLSC-EVs populations were electroporated with the siRNA PCS-C2 using 2 pulses of 20 ms, (p-value>0.05) (biological activity reduction of −0.75% and −2.04%, respectively). Compared to controls, a decrease in biological activity corresponding to 7% was determined as a “functional” threshold, meaning that a population of exogenous molecule-loaded EVs which exhibits a reduction in biological activity>7% is to be considered functionally damaged (Table 3). Moreover, the tubulogenesis test carried out by the present inventors showed that unloaded MSC-EVs and HLSC-EVs induced an increase in vessel formation of 1.49 fold and 1.13 fold, respectively, in comparison to untreated control endothelial cells. Overall, these data indicate that selected electroporation conditions, i.e. low number of pulses, provide a loading method for exogenous molecules into adult stem cell EVs which surprisingly preserve EVs integrity and functionality.

TABLE 3
Reduction of biological activity of loaded adult stem EVs
relative to unloaded controls
Biological activity reduction (%) in respect to unloaded EVs

| | MSC | MSC | | HLSC | |
|---|---|---|---|---|---|
| MSC | 10 p | 2 p | HLSC | 10 p | HLSC2 p |
| 0.00% | 12.81% | −0.75% | 0.00% | 7.29% | −2.04% |

1. A composition comprising a population of extracellular vesicles (EVs), wherein the EVs of the population are loaded with an exogenous molecule and are not damaged, wherein absence of damage is defined as follows:
(i) the mean diameter in the population of exogenous molecule-loaded EVs is increased by no more than 10% compared to the mean diameter in a population of unloaded control EVs;
(ii) the total nucleic acid content present in the population of exogenous molecule-loaded EVs is not significantly decreased compared to the total nucleic acid content present in the population of unloaded control EVs; and/or
(iii) the mean expression level of a panel of surface markers in the population of exogenous molecule-loaded EVs is reduced by no more than 15% compared to the mean expression level of the same panel of surface markers in the population of unloaded control EVs.

2. The composition according to claim 1, wherein the EVs are derived from a stem cell, preferably from an adult stem cell.

3. The composition according to claim 2, wherein the stem cell is an adult stem cell selected from a human liver stem cell (HLSC) and a human mesenchymal stem cell (MSC).

4. The composition according to claim 1, wherein the EVs are derived from a biological fluid, a conditioned cell medium or a tissue culture medium.

5. The composition according to claim 4, wherein the biological fluid is whole blood, plasma, serum or urine.

6. The composition according to claim 1, wherein the exogenous molecule is selected from the group consisting of nucleic acid, protein, peptide, aptamer, chemical drug and any combination thereof.

7. The composition according to claim 1, wherein the expression level of the panel of surface membrane-markers in the population of exogenous molecule-loaded EVs is reduced by no more than 12%.

8. The composition according to claim 1, wherein biological activity in the population of exogenous molecule-loaded EVs is not significantly reduced compared to the biological activity in the population of unloaded control EVs.

9. The composition according to claim 8, wherein the biological activity is a pro-angiogenic activity.

10. The composition according to claim 1, wherein the amount of exogenous molecules loaded in the extracellular vesicles is of at least 3 ng/10¹⁰ EVs.

11. The composition according to claim 1, wherein the loaded exogenous molecule is a nucleic acid.

12. The composition according to claim 11, wherein loaded nucleic acid is a microRNA (miRNA) or a small interfering RNA (siRNA).

13. The composition according to claim 12, wherein the miRNA and/or the siRNA is a pro-angiogenic RNA, an anti-angiogenic RNA or an anti-tumor RNA.

14. The composition according to claim 12 or 13, wherein the miRNA is hsa-miR-451 and/or hsa-miR-31.

15. A method for treating, in a subject in need thereof, a disease selected from the group consisting of cancer disease, cardiovascular disease, genetic disease, fibrotic diseases, wound healing, organ injury and viral infection, the method comprising administering to said subject the composition of claim 11.

16. The composition according to claim 1, wherein the panel of surface markers comprises one or more markers selected from the group consisting of CD9, CD19, CD81, CD86, CD90, HLA DR, CD47, CD34, CD40, CD31, CD144, CD3, CD146, CD105, CD5, HLA ABC, CD29, CD44, CD49d, CD49e, CD49f.

17. The composition according to claim 1, wherein exogenous molecule loading is performed by electroporation.

18. The composition according to claim 1, wherein the composition is obtainable by electroporation.

19. The composition according to claim 18, wherein electroporation is carried out at a voltage comprised between 500 and 900 Volt, with a number of pulses comprised between 1 and 10, the duration of each pulse being comprised between 10 and 25 milliseconds.

20. The composition according to claim 19, wherein electroporation is carried out at a voltage comprised between 600 and 800 Volt.

21. The composition according to claim 19, wherein the duration of each pulse is comprised between 18 and 22 milliseconds.

22. The composition according to claim 19, wherein electroporation is carried out with 2 or 10 pulses.

23. A method of loading a population of extracellular vesicles (EVs) with an exogenous molecule, the method comprising subjecting the EVs of the population to electroporation, wherein electroporation is carried out at a voltage comprised between 500 and 900 Volt, with a number of pulses comprised between 1 and 10, the duration of each pulse being comprised between 10 and 25 milliseconds.

24. The method according to claim 23, wherein the EVs are derived from a stem cell, preferably from an adult stem cell.

25. The method according to claim 23, wherein the EVs are derived from a biological fluid, a conditioned cell medium or a tissue culture medium.
